(19) United States
(12) Patent Application Publication
(10) Pub. No.: US 2011/0086366 A1
(43) Pub. Date: Apr. 14, 2011

(54) METHODS FOR ASSESSING THE RISK OF ADVERSE EVENTS UPON TREATMENT WITH IGG4 ANTIBODIES

(75) Inventors: Aran Frank Labrijn, Nigtevecht (NL); Janine Schuurman, Diemen (NL); Jan Van De Winkel, Zeist (NL); Paul Parren, Utrecht (NL)

(73) Assignee: GENMAB A/S, Copenhagen (DK)

(21) Appl. No.: 12/918,768

(22) PCT Filed: Feb. 20, 2009
(86) PCT No.: PCT/EP09/52044

§ 371 Date: Dec. 22, 2010

(30) Foreign Application Priority Data
Feb. 22, 2008 (DK) PA2008 00256

(51) Int. Cl. G01N 33/53

(52) U.S. Cl. 435/7.21; 436/501

(57) ABSTRACT

The invention relates to methods and kits for assessing the risk, for an individual, of developing an adverse event upon treatment with a therapeutic antibody which is capable of Fab-arm exchange, said method comprising the steps of: a) providing a sample from an individual who is a candidate for treatment with said therapeutic antibody, b) assaying said sample for the presence of circulating IgG4 antibodies that binds an antigen known or suspected to be associated with a causative agent of said adverse event, and c) assessing, on the basis of the outcome of the assay of step b), the risk that the individual will develop said adverse event upon treatment with the therapeutic antibody, wherein the risk of development of said adverse event increases with increased level of said circulating IgG4 antibodies.

### FIELD OF THE INVENTION

The present invention relates to methods and kits for assessing the risk of developing adverse events upon treatment with a therapeutic antibody which is capable of Fab-arm exchange, in particular therapeutic antibodies of the IgG4 isotype.

### BACKGROUND OF THE INVENTION

For the design of antibody-based therapeutics the choice of the antibody backbone has largely been governed by the distinct structural and functional properties of the individual immunoglobulin (sub)classes. IgG4 antibodies differ functionally from the other IgG subclasses in their anti-inflammatory activity, making them the preferred subclass for applications where recruitment of immune effector functions is unnecessary (e.g. if only targeted delivery of therapeutic conjugates is required) or even undesired (e.g. if only receptor blocking without cell depletion is desired).

IgG4 antibodies are capable of exchanging Fab arms by swapping a heavy chain and attached light chain (half molecule) with a heavy-light chain pair from another molecule, resulting in bispecific antibodies (1-4). This process, termed “Fab-arm exchange” herein, has been shown to occur under reducing conditions in vitro and in vivo in mice (4). The ability of IgG4 antibodies to undergo Fab-arm exchange has been accredited to the instable core-hinge sequence in combination with sequence determinants in the IgG4 CH3 domain (4). Replacement of core-hinge residue Ser228 by Pro (S228P) results in a partial stabilization of an IgG4 molecule in vitro and in vivo (1-4).

Natalizumab (Tysabri®), directed to the α4 subunit of α4β1 (VLA-4) and α4β7 integrins, and gemtuzumab (Mylotarg®), specific for CD33, are two humanized IgG4 antibodies currently approved for human use. Natalizumab is effective in the treatment of multiple sclerosis (MS) and gemtuzumab, conjugated to a cytotoxic calicheamicin derivative, is used to treat Acute Myeloid Leukemia (AML). Development of another humanized IgG4-based therapeutic, TGN1412 (CD28-specific), was discontinued after causing unforeseen adverse events in healthy individuals. Natalizumab has also been associated with adverse events, in particular progressive multifocal leukoencephalopathy, a central nervous system (CNS) infection with the JC polyoma virus.

Thus, while antibody-based therapy has significantly improved treatment and prognosis of a number of diseases, including chronic diseases, safety remains an important concern. There is therefore a need for improved methods of determining the risk of adverse events in connection with antibody-based treatment.

### SUMMARY OF THE INVENTION

It has now been found that in human patients undergoing therapy with an IgG4 antibody, there is Fab-arm exchange between the administered therapeutic antibody and endogenous circulating IgG4 antibodies of the patient. This results in the formation of a significant population of bispecific antibodies in the blood of the patient, consisting of bispecific antibodies which have a first specificity corresponding to the specificity of the administered therapeutic antibody and a second specificity, differing form the first specificity, directed against a different antigen.

If said second specificity is directed to an antigen which can mediate an adverse event, for example a viral antigen which can mediate a viral infection, then the formed bispecific antibody can potentially function as an undesired targeting vehicle which could target the antigen towards susceptible cells or tissues. For example, a bispecific antibody having a first specificity for a molecule found on a tissue which is susceptible to a viral infection, and a second specificity for the virus, could, upon contact with the virus, efficiently target the virus to the susceptible tissue, potentially resulting in a much higher rate of infection than if such a bispecific antibody was not present in the blood circulation of the patient. Seropositive individuals who have antibodies, specifically IgG4 antibodies, that binds an antigen that can mediate an adverse event, e.g. a virus, can obtain such bispecific antibodies upon treatment with the therapeutic antibody and therefore, such individuals are more at risk of developing the adverse event, e.g. the viral infection, than seronegative individuals, who cannot generate such bispecific antibodies upon treatment with the therapeutic antibody.

Accordingly, individuals who are seropositive for an antigen that can mediate an adverse event are more at risk of developing the adverse event upon treatment with a therapeutic IgG4 antibody than individuals who are seronegative. The determination of whether the individual is seropositive or seronegative for the antigen is therefore indicative of the risk of developing the adverse event upon treatment with an IgG4 antibody, or other antibody capable of undergoing Fab-arm exchange.

Thus, in a first aspect, it is an object of the present invention to provide a method of assessing the risk, for an individual, of developing an adverse event upon treatment with a therapeutic antibody which is capable of Fab-arm exchange, said method comprising the steps of:

a) providing a sample from an individual who is a candidate for treatment with said therapeutic antibody,

b) assaying said sample for the presence of circulating IgG4 antibodies that bind an antigen known or suspected to be associated with a causative agent of said adverse event, and

c) assessing, on the basis of the outcome of the assay of step b), the risk that the individual will develop said adverse event upon treatment with the therapeutic antibody, wherein the risk of development of said adverse event increases with increased level of said circulating IgG4 antibodies.

In a particular embodiment, the individual is a candidate for treatment with a therapeutic IgG4 antibody that binds VLA4, e.g. natalizumab, and the sample of said individual is tested for the presence of IgG4 antibodies that bind the JC virus in order to assess the risk that the individual will develop progressive multifocal leukoencephalopathy upon treatment with the anti-VLA4 antibody.

In a further main aspect, the invention relates to a method of assessing, for an individual who has been treated with a therapeutic antibody which is capable of Fab-arm exchange, the risk of developing an adverse event, said method comprising the steps of:

a) providing a sample from said individual,

b) assaying said sample for
1. the presence of circulating IgG4 antibodies that bind an antigen known or suspected to be associated with a causative agent of said adverse event,
or
2. the presence of bispecific antibodies having a first specificity corresponding to the specificity of the therapeutic antibody and a second specificity directed against an antigen known or suspected to be associated with a causative agent of said adverse event, and

c) assessing the risk of development an adverse event on the basis of the outcome of the assay of step b), wherein the presence of said bispecific antibodies indicates an increased risk of development of an adverse event.

In an even further aspect, the invention relates to a kit comprising
a) one or more of the materials required for performing the method of the invention, and
b) instructions describing or referring to the method of the invention.

In a yet further aspect, the invention relates to a kit comprising:
a) an anti-human IgG4 antibody, and
b) an antigen known or suspected to be associated with a causative agent of an adverse event, preferably an antigen of an infectious agent, more preferably a virus particle or an antigen of a virus, such as a JC virus particle or an antigen of a JC virus.

### BRIEF DESCRIPTION OF THE DRAWINGS

FIG. 1 Loss of half-molecules under non-reducing conditions in core-hinge stabilized IgG4 antibody therapeutics. (Therapeutic) IgG4 and control molecules were analyzed on a non-reducing SDS-polyacrylamide gel. The molecular sizes of intact antibodies (H2L2), half-molecules (HL), heavy-chains (H) and light-chains (L) are indicated.

FIG. 2 Core-hinge stabilization protects IgG4 antibody therapeutics from Fab-arm exchange in vitro. (a-h) Mixtures of IgG4-CD20/IgG4-EGFR (a,e), IgG4-CD20/IgG4S228P-EGFR (b,f), IgG4-CD20/natalizumab (c,g) and IgG4-CD20/gemtuzumab (d,h) were incubated for 24 hours in the absence (a-d) or presence (e-h) of 0.5 mM GSH. Antibody mixtures were subsequently deglycosylated with peptide N-glycosidase F, and analyzed by ESI-TOF mass spectrometry. Deconvoluted ESI-TOF spectra are shown. (i-I) Additionally, bispecificity was directly visualized by ELISA, for the mixtures of IgG4-CD20/IgG4-EGFR (i) and IgG4-CD20/IgG4S228P-EGFR (j), and flow cytometry, for the mixtures of IgG4-CD20/natalizumab (k) and IgG4-CD20/gemtuzumab (I). Antibody mixtures incubated in the absence (open black symbols) or presence of 0.5 mM GSH (closed black symbols) or 5 mM GSH (closed gray symbols) are indicated. Binding of gemtuzumab alone was used as control for CD33 expression (crosses).

FIG. 3 Core-hinge stabilization protects IgG4 antibody therapeutics from Fab-arm exchange in vivo. Groups (n=4) of SCID mice were injected with antibody mixtures (300 μg of each) of IgG4-CD20/IgG4-EGFR (closed circles), IgG4-CD20/IgG1-EGFR, IgG4-CD20/IgG4S228P-EGFR, IgG4-CD20/natalizumab (closed squares) and IgG4-CD20/gemtuzumab. The generation of bispecific antibodies was followed over time and quantified by ELISA and flow cytometry (see legend FIG. 2). Bispecific antibodies were quantified using an in vitro exchanged antibody mixture as reference. Data points represent mean±SEM values of four mice, measured at least twice in separate experiments. No bispecific antibodies could be detected in the IgG4-CD20/IgG1-EGFR, IgG4-CD20/IgG4S228P-EGFR and IgG4-CD20/gemtuzumab mixtures. The detection limit of the assays is indicated (dotted line) and represents serum levels of 2000 ng/ml.

FIG. 4 Patient information.

FIG. 5 Natalizumab exchanges Fab arms with patients' IgG4 during treatment (a) MS patients received three monthly doses of 300 mg natalizumab (black arrows). Plasma samples (grey arrows) were drawn before (T0) and after treatment (T2-T6; see also FIG. 4) for analysis. Bispecific antibodies were measured in the absence (b) or presence (c) of an excess of exogenous natalizumab. Statistical significance was determined by paired Student's t-test (*** p<0.001).

FIG. 6 Detection of Fab-arm exchanged natalizumab containing lambda light-chains. Mixtures of natalizumab/IgG4-637 (closed squares) and natalizumab/pooled human immunoglobulin (containing ˜3% IgG4; Sanquin) (closed circles) were incubated for 24 hours in the presence of 0.5 mM GSH. Fab-arm exchanged natalizumab was measured by flow cytometry by using PE-conjugated anti-human lambda light-chain for detection. Natalizumab only (open squares) was included as control. Representative results are shown.

FIG. 7 Analysis of patient plasma by size-exclusion chromatography. (a,b) Plasma samples from two representative natalizumab-treated MS patients was fractionated by size-exclusion chromatography (solid line). Bispecific antibodies were subsequently measured in individual fractions by flow cytometry (solid circles). Additionally, IgG4 levels were quantified in the individual fractions by ELISA (crosses). The analysis showed that bispecific IgG4 eluted within the monomeric IgG fractions.

### DETAILED DESCRIPTION OF THE INVENTION

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H} or VH) and a heavy chain constant region. The heavy chain constant region typically is comprised of three domains, C_{H}1, C_{H}2, and C_{H}3. Each light chain typically is comprised of a light chain variable region (abbreviated herein as V_{L} or VL) and a light chain constant region. The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each V_{H} and V_{L} is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)). Typically, the numbering of amino acid residues in this region is performed by the method described in Kabat et al., Sequences of Proteins of Immunological Interest, 5th Ed. Public Health Service, National Institutes of Health, Bethesda, Md. (1991) (phrases such as variable domain residue numbering as in Kabat or according to Kabat herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of V_{H} CDR2 and inserted residues (for instance residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “antibody” (Ab) in the context of the present invention refers to an immunoglobulin molecule, a fragment of an immunoglobulin molecule, or a derivative of either thereof, which has the ability to specifically bind to an antigen under typical physiological conditions with a half life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an Fc-mediated effector activity). The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation. An antibody may also be a bispecific antibody, diabody, or similar molecule (see for instance PNAS USA 90(14), 6444-8 (1993) for a description of diabodies).

As indicated above, the term antibody herein, unless otherwise stated or clearly contradicted by context, includes fragments of an antibody that retain the ability to specifically bind to the antigen. It has been shown that the antigen-binding function of an antibody may be performed by fragments of a full-length antibody. Examples of binding fragments encompassed within the term “antibody” include (i) a Fab′ or Fab fragment, a monovalent fragment consisting of the V_{L}, V_{H}, C_{L} and C_{H}1 domains, or a monovalent antibody as described in WO2007059782 (Genmab); (ii) F(ab′)₂ fragments, bivalent fragments comprising two Fab fragments linked by a disulfide bridge at the hinge region; (iii) a Fd fragment consisting essentially of the V_{H} and C_{H}1 domains; (iv) a Fv fragment consisting essentially of the V_{L} and V_{H} domains of a single arm of an antibody, (v) a dAb fragment (Ward et al., Nature 341, 544-546 (1989)), which consists essentially of a V_{H} domain and also called domain antibodies (Holt et al; Trends Biotechnol. 2003 November; 21(11):484-90); (vi) camelid or nanobodies (Revets et al; Expert Opin Biol Ther. 2005 January; 5(1):111-24) and (vii) an isolated complementarity determining region (CDR). Furthermore, although the two domains of the Fv fragment, V_{L} and V_{H}, are coded for by separate genes, they may be joined, using recombinant methods, by a synthetic linker that enables them to be made as a single protein chain in which the V_{L} and V_{H} regions pair to form monovalent molecules (known as single chain antibodies or single chain Fv (scFv), see for instance Bird et al., Science 242, 423-426 (1988) and Huston et al., PNAS USA 5879-5883 (1988)). Such single chain antibodies are encompassed within the term antibody unless otherwise noted or clearly indicated by context. Although such fragments are generally included within the meaning of antibody, they collectively and each independently are unique features of the present invention, exhibiting different biological properties and utility. These and other useful antibody fragments in the context of the present invention are discussed further herein. It also should be understood that the term antibody, unless specified otherwise, also includes polyclonal antibodies, monoclonal antibodies (mAbs), antibody-like polypeptides, such as chimeric antibodies and humanized antibodies, and antibody fragments retaining the ability to specifically bind to the antigen (antigen-binding fragments) provided by any known technique, such as enzymatic cleavage, peptide synthesis, and recombinant techniques.

As used herein, “isotype” refers to the immunoglobulin class (for instance IgG1, IgG2, IgG3, IgG4, IgD, IgA, IgE, or IgM) that is encoded by heavy chain constant region genes.

As used herein, the term “binding” in the context of the binding of an antibody to a predetermined antigen typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BIAcore 3000 instrument using the antigen as the ligand and the antibody as the analyte, and binds to the predetermined antigen with an affinity corresponding to a K_{D} that is at least ten-fold lower, such as at least 100 fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The amount with which the affinity is lower is dependent on the K_{D} of the antibody, so that when the K_{D} of the antibody is very low (that is, the antibody is highly specific), then the amount with which the affinity for the antigen is lower than the affinity for a non-specific antigen may be at least 10,000 fold.

The term “k_{d}” (sec⁻¹), as used herein, refers to the dissociation rate constant of a particular antibody-antigen interaction. Said value is also referred to as the k_{off} value.

The term “kₐ” (M⁻¹×sec⁻¹), as used herein, refers to the association rate constant of a particular antibody-antigen interaction.

The term “K_{D}” (M), as used herein, refers to the dissociation equilibrium constant of a particular antibody-antigen interaction.

The term “K_{A}” (M⁻¹), as used herein, refers to the association equilibrium constant of a particular antibody-antigen interaction and is obtained by dividing the kₐ by the k_{d}.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “bispecific antibody” is intended to include any antibody, which has two different binding specificities, i.e. the antibody binds two different epitopes, which may be located on the same target antigen or, more typically, on different target antigens.

“Treatment” refers to the administration of an effective amount of a therapeutically active compound with the purpose of easing, ameliorating, arresting or eradicating (curing) symptoms or disease states.

An “effective amount” refers to an amount effective, at dosages and for periods of time necessary, to achieve a desired therapeutic result. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects.

The term “individual” when used herein refers to a human being.

When used herein the term “therapeutic antibody which is capable of Fab-arm exchange” refer to a therapeutic antibody which is capable of undergoing Fab-arm exchange (half-molecule exchange) in vivo in humans. A typical example of such an antibody is an antibody of the IgG4 isotype. However, alternatively, it may be an antibody of another isotype which has been modified so that it is capable of undergoing Fab-arm exchange. For example, it has been shown that an IgG1 antibody which has been modified in the CH3 region, e.g. an IgG1 antibody in which the CH3 region has been replaced by a CH3 of IgG4, can undergo Fab-arm exchange. The ability to undergo Fab arm exchange can be tested in vivo or in vitro under reducing conditions (4).

### Methods of the Invention

In a first main aspect, the invention relates to a method of assessing the risk, for an individual, of developing a particular adverse event upon treatment with a therapeutic antibody which is capable of Fab-arm exchange, said method comprising the steps of:

a) providing a sample from an individual who is a candidate for treatment with said therapeutic antibody,

b) assaying said sample for the presence of circulating IgG4 antibodies that bind an antigen known or suspected to be associated with a causative agent of said adverse event, and

c) assessing, on the basis of the outcome of the assay of step b), the risk that the individual will develop said adverse event upon treatment with the therapeutic antibody, wherein the risk of development of said adverse event increases with increased level of said circulating IgG4 antibodies.

The individual for whom the risk of an adverse event is being assessed in the method of the invention may be any individual who is a candidate for treatment with a particular therapeutic antibody capable of Fab-arm exchange.

In one embodiment, the individual is an immunocompromised individual.

The adverse event for which the risk is assessed may be any adverse, i.e. undesired, event that may occur in connection with the antibody treatment. In one embodiment, the adverse event is an infectious disease, such as a viral, bacterial, fungal or parasitic disease. For example, the viral disease may be a disease caused by a JC virus, such as progressive multifocal leukoencephalopathy or a disease caused by dengue virus, such as dengue hemorrhagic fever or dengue shock syndrome.

In one embodiment of the method of the invention, the therapeutic antibody which is capable of Fab exchange is an IgG4 antibody, e.g. a humanized, chimeric or human IgG4 antibody.

In one embodiment, the adverse event is an infectious disease and the therapeutic antibody is an antibody that binds a molecule which is present on cells or tissues that are susceptible of being infected by said infectious agent or a molecule which is present on cells that are capable of transporting said infectious agent to a susceptible tissue, i.e. a tissue which upon infection will result in the adverse event.

In one embodiment, the therapeutic antibody binds a molecule selected from the group consisting of: an integrin subunit, such as VLA-4, low density lipoprotein (LDL) receptors, αvβ1, αvβ3, αMβ2, αvβ5, CAR (coxsackie and adenovirus receptors), CD21, heperan sulfate, Hve A, Hve B, Hve C, TNFSF14, HVEM, Prr1, Prr2, Nectin-1, Nectin-2, β1, β2Microglobulin/MHC I, α3β1, αIIbβ3, sialic acid residues, gangliosides, CD46, moesin, erythrocyte P antigen, alpha 2-6 sialic acid residue, serotonergic receptors (5HT2aR), alpha 2-6 sialic acid residue, CD44, CD155 (PVR), ICAM-1, α2β1 (VLA-2), αvβ3, α5β1, αvβ3, αvβ6, decay accelerating factor, EGF receptor, αxβ2, α2β1, α4β7, CD4, CCR5, CXCR4, galactosylceramide, CCR3, phosphate permease, acetylcholine receptor, phospholipids, NCAM, NGFR, phosphatidyl serine, laminin receptors, HLA H2-K, H2-D, lactate dehydrgenase Ia, CEA, EGFR, CD105, CD33, CD15, FcgRI and FcgRII.

In further embodiments, the adverse event is caused by a virus and the circulating IgG4 antibodies tested for in step b) bind an antigen of said virus, wherein the virus and the molecule bound by the therapeutic antibody are selected from the combinations shown in Table 1.

TABLE 1

| Adverse event caused by: | Therapeutic antibody binds: |
|---|---|
| Adenovirus | αvβ1, αvβ3, αMβ2 or αvβ5, |
| | CAR (coxsackie and adenovirus receptors) |
| Epstein Bar Virus (EBV) | CD21 |
| Herpes Simplex Virus (HSV) | Heperan sulfate, αvβ3, Hve A, Hve B, Hve C |
| | TNFSF14, HVEM, Prr1, Prr2, Nectin-1 or Nectin-2 |
| Human Cytomegalovirus | β1, αvβ3, Heperan sulfate or β2Microglobulin/MHC I |
| Human Herpes Virus | α3β1 or α2β1 |
| Sin Nombre Virus | αIIβ3 or αvβ3 |
| Prospect Hill Virus | β1 |
| Influenza virus | Sialic acid residues |
| Sendai virus | Gangliosides |
| Measles virus | CD46 or Moesin |
| B19 | Erythrocyte P antigen |
| JC virus | alpha 2-6 sialic acid residue or serotonergic receptors |
| | (5HT2aR) |
| BK human polyomavirus | alpha 2-6 sialic acid residue or gangliosides |
| Polio virus | CD44 or CD155 (PVR) |
| Rhinovirus | ICAM-1 |
| Echovirus | α2β1 (VLA-2) or αvβ3 |
| Foot-and-mooth disease virus | α5β1 or αvβ3 |
| Coxsackievirus | αvβ3, αvβ6, decay accelerating factor or CAR |
| Vaccinia virus | EGF receptor |
| Reovirus | β1, sialic acid residues or EGF receptor |
| Rotavirus | αxβ2, α2β1, αvβ1, α4β7, αvβ3, gangliosides or sialic |
| | acid residues |
| Human Immunodeficiency Virus | CD4, CCR5, CXCR4, galactosylceramide or CCR3 |
| Gross leukemia virus | Phosphate permease |
| Rabies virus | acetylcholine receptor, gangliosides, phospholipids, |
| | NCAM or NGFR |
| Vesicular stomatitis virus | phosphatidyl serime |
| Sindbis virus | Laminin receptors |
| Semliki Forest virus | HLA H2-K, H2-D or lactate dehydrgenase Ia |
| Adenovirus | CEA, EGFR or CD105 |
| Dengue | CD33, CD15, FcgRI or FcqRII |

In one embodiment of the method of the invention, the sample that is provided in step a) is a blood sample, such as a serum sample.

In step b) of the method of the invention, the sample is assayed for the presence of circulating IgG4 antibodies, i.e. IgG4 antibodies circulating in the body of the individual from who that sample was taken, that bind an antigen known or suspected to be associated with a causative agent of said adverse event. The assay can be qualitative (absence/presence) or quantitative detection.

The assay in step b) detects IgG4 antibodies that bind an antigen known or suspected to be associated with a causative agent of said adverse event. For example, if the adverse event is an infectious disease, the causative agent is an infectious agent, and the antigen may be an antigen of said infectious agent, for example, a surface-exposed antigen of an infectious agent, e.g. a viral envelope protein or a cell-surface exposed molecule of a bacterial or fungal cell.

The assay performed in step b) may be carried out using any standard method known in the art. For example, the assay may be an ELISA, wherein the antigen, e.g. a viral envelope protein, is coated on a solid support, and detection is performed using an anti-human-IgG4 antibody conjugated to a detectable label. In an alternative set up, the solid support is coated with anti-human-IgG4 antibody and the relevant circulating IgG4 antibodies are being detected using a conjugated antigen, e.g. a conjugated envelope protein.

In one particular embodiment of the method of the invention, the adverse event is progressive multifocal leukoencephalopathy and the antigen is an antigen of the JC virus. In a further embodiment, the adverse event is progressive multifocal leukoencephalopathy, the antigen is an antigen of the JC virus and the therapeutic antibody is antibody that binds VLA4, such as natalizumab. In further embodiments hereof, the individual is a patient suffering from multiple sclerosis, Crohn's disease or rheumatoid arthritis.

Assays for JC virus have for example been described in: Lundstig, A. and Dillner J. Serological diagnosis of human polyomavirus infection. Adv Exp Med Biol. 577:96-101 (2006) and Stolt et al. Seroepidemiology of the human polyomaviruses. J. Gen. Virol. 84:1499-1504 (2003)

In another particular embodiment, the adverse event is a disease caused by dengue virus, such as dengue hemorrhagic fever or dengue shock syndrome, and the antigen is an antigen of dengue virus. In a further embodiment hereof, therapeutic antibody is an antibody that binds Fc gamma RI, Fc gamma RII, beta2 microglobulin, CD15 or CD33.

Step c) of the method of the invention comprises an assessment, on the basis of the outcome of the assay of step b), of the risk that the individual will develop the adverse event upon treatment with the therapeutic antibody.

For example, if the individual has circulating IgG4 antibodies that bind an antigen known or suspected to be associated with a causative agent of an adverse event, e.g. anti-viral IgG4 antibodies, then bispecific antibodies will be generated upon treatment of said individual with the therapeutic antibody capable of Fab-arm exchange. As explained above, said bispecific antibodies may then target viral particles to a target tissue, thus increasing the infectivity of the virus, i.e. increasing the risk that the individual will develop the viral disease upon contact with the virus. Thus, the presence of circulating anti-viral IgG4 antibodies is indicative of a higher risk of viral disease upon treatment.

In some embodiments, the assessment in step c) may involve a comparison of the result of the assay in step b) with a cut-off value indicative of increased risk for development of the adverse event. For example, in some embodiments, a level of circulating IgG4 antibodies below a certain limit may not be correlated to higher risk and in such cases, an individual might not be considered to be a higher risk of developing the adverse event.

The assessment in step c) may lead to a decision as to whether said individual who is candidate for treatment with the therapeutic antibody should indeed be treated with the antibody or whether alternative medication should be used or additional precautionary measures should be taken to prevent or treat the adverse event.

In embodiments of the method of the invention wherein the adverse event is an infectious disease, it may sometimes be useful, as a further step, also to determine the presence of said infectious agent in a sample from said individual.

In a further main aspect, the invention relates to a method of assessing, for an individual who has been treated with a therapeutic antibody which is capable of Fab-arm exchange, the risk of developing an adverse event, said method comprising the steps of:

a) providing a sample from said individual,

b)—assaying said sample for the presence of circulating IgG4 antibodies that bind an antigen known or suspected to be associated with a causative agent of said adverse event,

or

assaying said sample for the presence of bispecific antibodies having a first specificity corresponding to the specificity of the therapeutic antibody and a second specificity directed against an antigen known or suspected to be associated with a causative agent of said adverse event

c) assessing the risk of development an adverse event on the basis of the outcome of the assay of step b), wherein the presence of said bispecific antibodies indicates an increased risk of development of an adverse event.

This method may comprise one or more of the additional features described above.

In this method, the individual has been treated with said therapeutic antibody, and thus, if the individual had circulating IgG4 antibodies that bind said antigen, the individual will in his circulation presumably have obtained or obtain bispecific antibodies derived from the therapeutic antibody. Assaying a sample from such an individual for circulating IgG4 antibodies that bind said antigen may still be useful e.g. to assess whether additional precautionary measures should be taken to prevent or treat the adverse event. Instead of assaying the sample for circulating IgG4 antibodies that bind said antigen, it is also possible to assay directly for the presence of bispecific antibodies having a first specificity corresponding to the specificity of the therapeutic antibody and a second specificity directed against an antigen known or suspected to be associated with a causative agent of said adverse event. This may e.g. be done using ELISA assays analogous to those described in Example 3 herein.

### Kits of the Invention

In a further main aspect, the invention relates to a kit comprising:
a) one or more of the materials required for performing the method of the invention as described herein, and
b) instructions describing or referring to the method of the invention.

In one embodiment, said kit comprises an anti-human IgG4 antibody, optionally coated on a solid support or conjugated to a directly or indirectly detectable label.

In another embodiment, said kit comprises the antigen known or suspected to be associated with a causative agent of the adverse event, optionally coated on a solid support or conjugated to a directly or indirectly detectable label.

In a further embodiment, said kit comprises an anti-human IgG4 antibody, optionally coated on a solid support or conjugated to a directly or indirectly detectable label, and the antigen known or suspected to be associated with a causative agent of the adverse event, optionally coated on a solid support or conjugated to a directly or indirectly detectable label.

In a further main aspect, the invention relates to a kit comprising:
a) an anti-human IgG4 antibody, and
b) the antigen known or suspected to be associated with a causative agent of the adverse event
   In a preferred embodiment, one of a) and b) is conjugated to a directly or indirectly detectable label. In a further preferred embodiment, one of a) and b) is conjugated to a directly or indirectly detectable label, and the other is coated to solid support.

Preferably, the antigen is an antigen of an infectious agent, more preferably a virus particle or an antigen of a virus, such as a JC virus particle or an antigen of a JC virus. In one embodiment, the kit further comprises instructions describing or referring to the method of the invention as described herein.

Reagents included in the kits of the invention may include, for example, fluorescent tags, enzymatic tags, or other detectable tags. The reagents may also include secondary or tertiary antibodies or reagents for enzymatic reactions, wherein the enzymatic reactions produce a product that may be visualized.

In kits, an antibody is often provided in a lyophilized form in a container, either alone or in conjunction with additional antibodies specific for a target cell or peptide. Typically, a carrier (e.g., an inert diluent) and/or components thereof, such as a Tris, phosphate, or carbonate buffer, stabilizers, preservatives, biocides, biocides, inert proteins, e.g., serum albumin, or the like, also are included (often in a separate container for mixing) as well as additional reagents (also often in separate container(s)).

In certain kits, a secondary antibody is also included. The second antibody is typically conjugated to a label. In one example, reagent of the kit such as an antigen or antibody, may be added to nitrocellulose, or other solid support which is capable of immobilizing cells, cell particles, or soluble proteins. The support may then be washed with suitable buffers followed by treatment with the detectably labeled antigen or antibody. The solid phase support may then be washed with the buffer a second time to remove unbound antigen or antibody. The amount of bound label on the solid support may then be detected by known method steps.

Linked enzymes that react with an exposed substrate may be used to generate a chemical moiety which may be detected, for example, by spectrophotometric, fluorometric or by visual means, in the context of a antigen/antibody conjugate and/or fusion protein. Enzymes which may be used include malate dehydrogenase, staphylococcal nuclease, delta-5-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase, and acetylcholinesterase. It is also possible to label antigen or antibody with a fluorescent compound. When the fluorescent labeled antibody is exposed to light of the proper wave length, its presence may be detected due to fluorescence. Among the most commonly used fluorescent labeling compounds are fluorescein isothiocyanate, rhodamine, phycoerythrin, phycocyanin, allophycocyanin, o-phthaldehyde, and fluorescamine.

The antigen or antibodies included within the kit of the invention, may also be detectably labeled using fluorescence-emitting metals such as ¹⁵²Eu, or others of the lanthanide series. These metals may be attached to an antibody, for example, using such metal chelating groups as diethylenetriaminepentaacetic acid (DTPA) or ethylenediaminetetraacetic acid (EDTA).

Antigen or antibodies included within the kit may also be detectably labeled by coupling to a chemiluminescent compound. The presence of the chemiluminescently labeled antigen or antibody is then determined by detecting the presence of luminescence that arises during the course of a chemical reaction. Examples of particularly useful chemiluminescent labeling compounds are luminol, isoluminol, theromatic acridinium ester, imidazole, acridinium salt, and oxalate ester.

Likewise, a bioluminescent compound may be used to label an antigen or antibody. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase, and aequorin.

Detection of a labeled peptide or antibody, antibody fragment or derivative may be accomplished by a scintillation counter, for example, if the detectable label is a radioactive gamma emitter, or by a fluorometer, for example, if the label is a fluorescent material. In the case of an enzyme label, the detection may be accomplished by colorimetric methods which employ a substrate for the enzyme. Detection may also be accomplished by visual comparison of the extent of enzymatic reaction of a substrate in comparison with similarly prepared standards.

These methods and kits may be used to screen any suitable material. Examples of materials that may be screened include, for example, blood, serum, lymph, urine, inflammatory exudate, cerebrospinal fluid, amniotic fluid, a tissue extract or homogenate, and the like. However, the present invention is not limited to assays using only these samples, it being possible for one of ordinary skill in the art to determine suitable conditions which allow the use of other samples.

### REFERENCES

1. Angal, S. et al. A single amino acid substitution abolishes the heterogeneity of chimeric mouse/human (IgG4) antibody. Mol Immunol 30, 105-8 (1993).
2. Bloom, J. W., Madanat, M. S., Marriott, D., Wong, T. & Chan, S. Y. Intrachain disulfide bond in the core hinge region of human IgG4. Protein Sci 6, 407-15 (1997).
3. Schuurman, J., Perdok, G. J., Gorter, A. D. & Aalberse, R. C. The inter-heavy chain disulfide bonds of IgG4 are in equilibrium with intra-chain disulfide bonds. Mol Immunol 38, 1-8 (2001).
4. van der Neut Kolfschoten, M. et al. Anti-inflammatory activity of human IgG4 antibodies by dynamic Fab arm exchange. Science 317, 1554-7 (2007)
5. Lundstig, A. amd Diliner J. Serological diagnosis of human polyomavirus infection. Adv Exp Med Biol. 577:96-101 (2006)
6. Stolt et al. Seroepidemiology of the human polyomaviruses. J. Gen. Virol. 84:1499-1504 (2003)

### EXAMPLES

### Example 1

### Materials and Experimental Procedures

### Patient Samples

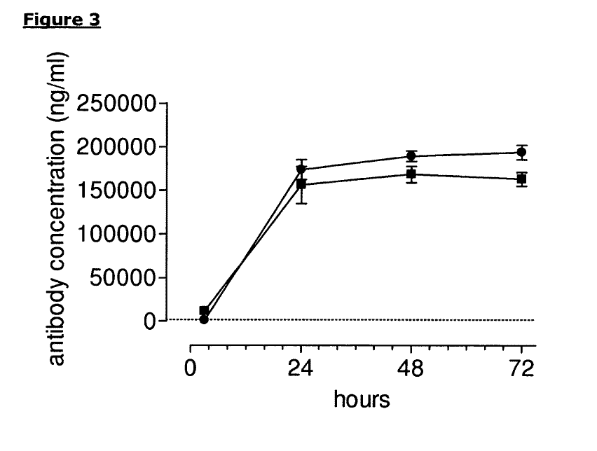

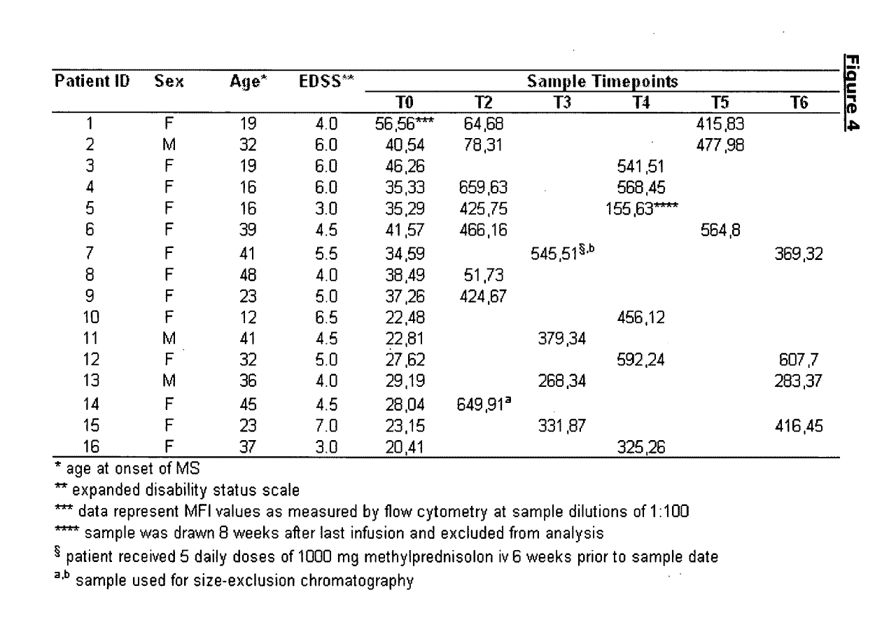

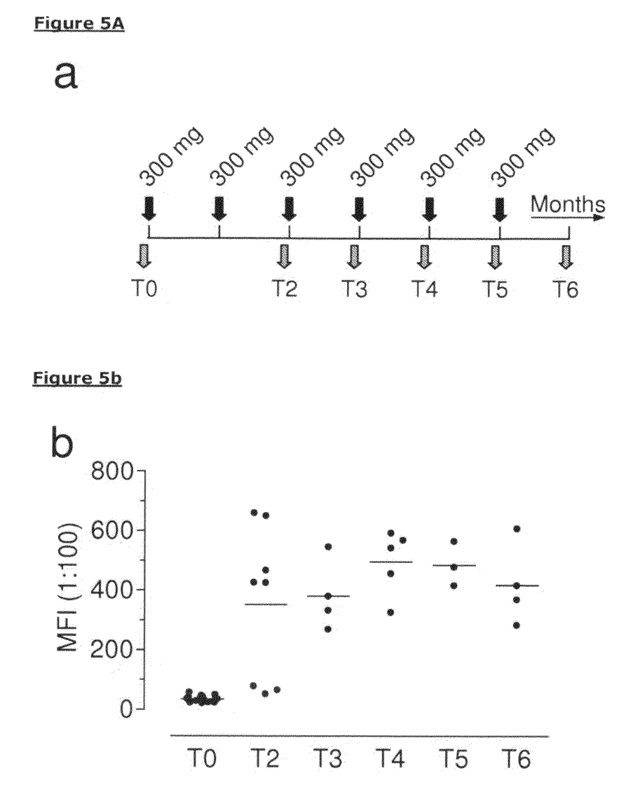

Plasma samples from MS patients starting natalizumab treatment were drawn under informed consent. Patients received natalizumab (at a dose of 300 mg) by intravenous infusion every 4 weeks. Blood samples were obtained before the start of therapy (T0; n=16) and at different time-points after subsequent infusions (T2-T6; see FIGS. 4 and 5). Sample drawing was done 4 weeks after the last infusion, just prior to the next infusion.

### Cell Lines

Jurkat (human T-cell leukemia) and HL-60 (human acute myelogenous leukemia) cells were obtained from the American Type Culture Collection (ATCC) and National Institute of Health Science (NIHS), respectively. Both cell lines were cultured in RPMI-1640 medium (Lonza) supplemented with 10% heat-inactivated fetal bovine serum (Hyclone), 50 IU/ml penicillin and 50 μg/ml streptomycin. HEK-293F cells (Invitrogen) were cultured in Freestyle medium (Invitrogen). CHO-K1SV cells (Lonza) were cultured in HAM's F12 (Invitrogen), supplemented with 10% fetal bovine serum (Bodinco).

### Commercial Antibodies

Natalizumab (Tysabri®, humanized IgG4κ), a monoclonal antibody directed to the o4 subunit of α4β1 and α4β7 integrins, and gemtuzumab (Mylotarg®, humanized IgG4κ conjugated to a calicheamicin derivative), a monoclonal antibody against CD33, were obtained from Biogen Idec/Elan Pharmaceuticals and Wyeth Pharmaceuticals, respectively. Pooled human immunoglobulin (Immunoglobulin I.V.; IVIG) was obtained from Sanquin and contained ˜3% IgG4 (of total IgG).

Cloning and Production of Antibodies

Construction of expression vectors for IgG1-EGFR, IgG4-EGFR, IgG1-CD20 and IgG4-CD20 has been described previously (4). In short, VH and VL coding regions of EGFR-specific HuMab 2F8 and CD20-specific HuMab 7D8, were cloned in expression vector pConG1f (Lonza) for the production of IgG1 heavy chain, and in pConKappa for the production of light chain. This yielded the vectors pConG1f2F8, pConG1f7D8, pConKappa2F8 and pConKappa7D8. For the production of IgG4 heavy chains, the VH regions of pConG1f2F8 and pConG1f7D8 were removed from these vectors by a HindIII/ApaI digestion and inserted into HindIII/ApaI digested pTomG4 vector, resulting in pTomG42F8 and pTomG47D8, respectively. Site directed mutagenesis was used to introduce the S228P (EU numbering) mutation in the hinge of IgG4 using pTomG42F8 as a template. A Quickchange site-directed mutagenesis kit (Stratagene) was used to Create the pTomG42F8CPPCNew vector.

All IgG1 and IgG4 antibodies were produced under serum-free conditions (Freestyle medium) by cotransfecting relevant heavy and light chain expression vectors in HEK-293F cells using 293fectin according to the manufacturer's instructions (Invitrogen). The IgG4S228P-EGFR was produced by cotransfecting relevant heavy and light chain expression vectors in CHO-K1SV cells using Lipofectin (Invitrogen) according to the manufacturer's instructions.

The following vectors were co-expressed: 1) pConG1f2F8 and pConKappa2F8 to produce IgG1-EGFR, 2) pTomG42F8 and pConKappa2F8 to produce IgG4-EGFR, 3) pTomG42F8CPPCNew and pConKappa2F8 to produce IgG4S228P-EGFR, and 4) pTomG47D8 and pConKappa7D8 to produce IgG4-CD20.

All IgG1, IgG4 and IgG4S228P antibodies were purified by Protein A affinity chromatography (rProtein A FF, GE Healthcare), dialysed overnight to PBS and filtered-sterilized over 0.2 μM dead-end filters. Concentration of purified IgGs was determined by nephelometry and absorbance at 280 nm. Purified proteins were analyzed by SDS-PAGE (see below), mass spectrometry and glycoanalysis.

### Cloning and Production of IgG4-637

Construction of expression vectors for IgG4-637 has been described previously¹. In short, VH and VL coding regions of acetylcholine receptor (AChR)-specific Fab 637² were cloned in expression vector pIgG1³ to yield pIgG1-637. The VH and VL coding sequences were subsequently cloned into pTomG4, for the production of IgG4 heavy chain, and pConLam2 (Lonza), for the production of light chain, respectively. This yielded the vectors pTomG4MG and pConLamMG that were co-expressed in CHO-K1SV cells to produce IgG4-637. Stable clones were selected after selection with 50 μM MSX. IgG4-637 was purified and analyzed as described in main text.

### ESI-TOF

Mixtures of natalizumab or gemtuzumab and IgG4-CD20 (200 μg/ml of each) were incubated for 24 hrs in the absence or presence of GSH (see below) and evaluated by electrospray ionization time-of-flight (ESI-TOF) mass spectrometry. Fifty μl samples containing the antibody mixtures were deglycosylated overnight with 1 μl H-glycosidase F (Roche Diagnostics). Samples were desalted on an Acquity UPLC™ (Waters) with a BEH C8, 1.7 μm, 2.1×50 mm column at 60° C. Five μl was injected and eluted with a gradient from 5% to 95% acetronitril (LC-MS grade; Biosolve) in de-ionized water (Millipore). The gradient contained 0.05% formic acid as organic modifier (Fluka). ESI-TOF mass spectra were recorded on-line on a microTOFTM mass spectrometer (Bruker) operating in the positive ion mode. In each analysis, a 500-5000 m/z scale was internally calibrated with ES tuning mix (Agilent Technologies). Mass spectra were deconvoluted using the Maximum Entropy algorithm, provided in DataAnalysis™ software v3.3 (Bruker).

### Trypsin Digestion

Each antibody sample (1 mg) was denatured in 400 μl Rapigest™ (Waters, Milford, Mass.) 0.1% containing 50 mM ammonium bicarbonate (Fluka BioChemika, Buchs, Switzerland) pH 8.0. Subsequently the samples were reduced by adding 3 μl dithiotreitol (DTT) 1.0 M and incubated for 30 min at 60° C. The denatured and reduced samples were alkylated with a 7 μl aliquot of iodoacetamide (IAA) 1.0M (Sigma-Aldrich, Saint Louis, Mo.) and incubated for 45 min at room temperature in the dark. In order to terminate the alkylation reaction, 3 μl DTT 1.0M was added. The digestion was performed overnight using trypsin (Promega, Madison, Wis.) at an enzyme/protein ratio of 1:50 (w/w). The digestion was derminated by adding trifluoroacetic acid (TFA)(Fluka BioChemika, Buchs, Switzerland) to a concentration of approximately 0.5% v/v. The samples were incubated for 45 minutes at 37° C. Subsequently, the acid treated samples were centrifuged at 13,000 rpm for 10 min and the supernatant was carefully transferred to the UPLC vial.

### UPLC Separation of Tryptic Peptides

The tryptic peptides were separated on a Aquity UPLC 2.1×150 mm BEH C18 column, particle size 1.7 μm (Waters, Milford, Mass.) using a linear gradient from 4 to 37% B over 116 min. Solvent A was 0.05% formic acid (FA) in water, and solvent B was 0.05% FA in 100% Acetonitrile (Biosolve, Valkenswaard, The Netherlands). Before sample injection, the UPLC column was equilibrated with 4% solvent B. The column temperature was maintained at 60° C. The flow rate was 0.3 ml/min, and a total of 16 μg of antibody digest was injected onto the column for analysis.

### Mass Spectrometry Analysis of Tryptic Peptides

The UPLC was directly coupled to a Bruker MicrOTOF (Bruker Daltonics, Bremen, Germany) equipped with an electrospray ionization source. Prior to analysis a 600-2700 m/z scale was calibrated with ES Tuning Mix (Agilent Technologies, Santa Clara, Calif.) in the positive ion mode. The spray source was set at 5000V.

### SDS-PAGE

All antibodies were analysed on SDS-PAGE (4-12% Bis-Tris; Invitrogen) under non-reducing conditions at neutral pH according to the manufacturer's instructions. The gels were stained with Coomasie (Invitrogen) and digitally imaged using the GeneGenius (Synoptics).

### GSH-Mediated Fab Arm Exchange in Vitro

As described previously (4), combinations of antibodies were mixed and incubated with reduced glutathione (GSH; Sigma) at a final concentration of 50 μg/ml per antibody. The final concentration of GSH was 0.5 mM. The mixtures were incubated at 37° C. for 24 hours and samples were drawn in PBS-TB (PBS/0.05% Tween-20/1% BSA), in which (bi)specific IgG concentrations were measured.

### Fab Arm Exchange in Vivo

Female SCID mice (6-8 week old) were obtained from Charles River Laboratories (Maastricht, The Netherlands) and housed in a barrier unit of the Central Laboratory Animal Facility (Utrecht, The Netherlands). The mice were kept in filter-top cages with water and food provided ad libitum. All experiments were approved by the Utrecht University animal ethics committee.

Mixtures of antibodies (300 μg each per mouse) were administered to mice (n=4) and blood samples were drawn from the saphenal vein at 3 hrs, 24 hours, 48 hours and 72 hours after administration. Blood was collected in heparin-containing vials, which were kept on ice, and centrifuged (5 minutes at 10,000 g) to separate the plasma from cells. Plasma was transferred to a new vial and stored at −20° C. for determination of bispecific antibody levels.

### Binding Assay for the Detection of CD20/EGFR Bispecific Antibodies

The presence of CD20/EGFR bispecific antibodies was determined using a sandwich ELISA as described previously (4). In short, ELISA plates (Greiner bio-one) were coated overnight with 2 μg/ml of recombinant EGFR (extracellular domain) in PBS at 4° C. The plates were washed and incubated with serial diluted plasma samples (in PBS-TB) for 90 minutes at room temperature (RT) under shaking conditions (300 rpm). Next, the plates were washed and incubated with 2 μg/ml of mouse anti-idiotype monoclonal antibody 2F2 SAB1.1 (directed against HumAb-CD20; Genmab) diluted in PBS-TB for 75 minutes at RT. Bound bispecific antibodies were detected with HRP-labeled goat-anti-mouse IgG (Jackson ImmunoResearch) and ABTS substrate (Roche Diagnostics). The color development reaction was stopped by addition of an equal volume of oxalic acid (Riedel de Haen) and absorbance was measured at 405 nm. Bispecific antibodies in plasma samples were quantified by non-linear regression curve-fitting (GraphPad) using an in vitro exchanged antibody mixture as reference (with the assumption that the maximal expected concentration of bispecific IgG4 was 50% of total IgG4 concentration).

### Binding Assay for the Detection of Fab Arm Exchanged Natalizumab and Gemtuzumab

To determine the presence of natalizumab or gemtuzumab half-molecules as part of bispecific antibodies, samples were serial diluted in FACS buffer (PBS/1% BSA/0.05% (w/v) NaN3) and incubated with Jurkat cells (VLA-4+) or HL-60 cells (CD33+) for 30 minutes at 4° C. To detect CD20/VLA-4 or CD20/CD33 bispecific antibodies, cells were washed with ice-cold FACS buffer and incubated with 2 μg/ml of mouse anti-idiotype monoclonal antibody 2F2 SAB1.1 diluted in FACS buffer for 30 minutes at 4° C. Bound bispecific antibodies were detected using phycoerythrin (PE)-conjugated goat-anti-mouse IgG (Jackson ImmunoResearch). Bispecific antibodies in plasma samples were quantified by non-linear regression curve-fitting (GraphPad) using an in vitro exchanged antibody mixture as reference (with the assumption that the maximal expected concentration of bispecific IgG4 was 50% of total IgG4 concentration).

Alternatively, to detect Fab-arm exchanged natalizumab in patient samples, bound bispecific antibodies were visualized using PE-conjugated anti-human lambda light chain (Southern Biotech). Samples were analyzed by flow-cytometry on a FACSCaliber (BD Biosciences).

### Statistical Analysis

Data analysis was performed using GraphPad Prism for Windows, version 4.03 (GraphPad). Data sets were compared by using two-tailed paired Student t tests. Statistical significance was accepted when P<0.05.

### Size-Exclusion Chromatography

Samples (600 μl) were heat-inactivated (30 minutes 56° C.) and applied to a Superdex 200 XK 26/60 column (Amersham Biosciences), which was connected to a FPLC system (Amersham Biosciences). The column was first equilibrated in PBS followed by calibration with pooled human immunoglobulin (Sanquin) to determine the retention volumes of monomeric, dimeric and aggregated IgG. Fractions of 1 ml were collected and bispecific antibodies and IgG4 concentrations were determined by flow cytometry (see main text) and ELISA (see below), respectively.

### Quantitative IgG4 ELISA

IgG4 antibody concentrations in (fractionated) plasma samples were determined by sandwich ELISA. In short, ELISA plates were coated overnight with 1 μg/ml of mouse anti-human IgG4 (MH164-4; Sanquin) in PBS at 4° C. The plates were washed and incubated with diluted plasma samples (in PBS-TB) for 60 minutes at room temperature (RT) under shaking conditions (300 rpm). Bound antibodies were detected by HRP-labelled mouse anti-human IgG4 (MH164-4) and ABTS substrate (Roche Diagnostics). The color development reaction was stopped by addition of an equal volume of oxalic acid (Riedel de Haen) and absorbance was measured at 405 nm. IgG4 was quantified by non-linear regression curve-fitting (GraphPad) using purified human IgG4 (The Binding Site) as reference.

### Example 2

### Analysis of Natalizumab and Gemtuzumab

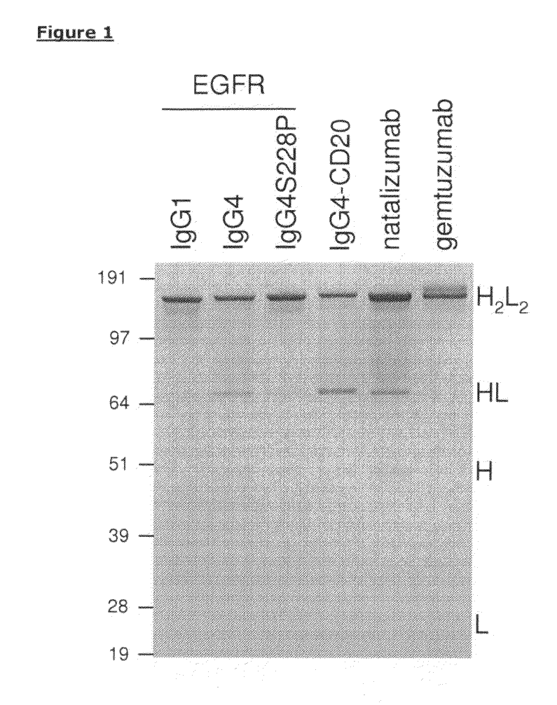

To determine the type of core-hinge samples were of natalizumab and gemtuzumab were analyzed by non-reducing SDS-PAGE and compared to a matched set (IgG1, IgG4 and IgG4S228P) of a human monoclonal antibody (HuMab) directed against the epidermal growth factor receptor (EGFR), HuMab 2F8, and a human IgG4 directed against CD20, HumAb 7D8. Whereas the IgG1 showed intact antibodies under non-reducing conditions, the IgG4 molecules revealed substantial amounts of half-molecules in addition to intact antibodies (FIG. 1). The S228P mutation (IgG4S228P) stabilized the IgG4 molecule as demonstrated by the loss of half-molecules. Analysis of natalizumab revealed the presence of half-molecules indicative of a wild-type IgG4 core-hinge. Gemtuzumab, however, showed no half-molecules, indicating a stabilized core-hinge, and additionally displayed two intact antibody bands, most likely representing the calicheamicin-conjugated and the naked antibody molecules (which are formulated as a 1:1 mixture as described in the Mylotarg® product information sheet). To confirm the hinge region amino acid sequences for natalizumab and gemtuzumab, samples were digested with CNBr and trypsin, reduced with DTT and analysed using on-line LC/ES-MS. A tryptic peptide with a mass of 944.08 Da was detected for natalizumab, corresponding to the theoretic mass ([M+3H]3+=943.8 Da) of the wild-type IgG4 peptide 219-YGPPCPSCPAPEFLGGPSVFLFPPKPK-248 (SEQ ID NO:1). For gemtuzumab a tryptic peptide of 947.42 Da was detected, corresponding to the theoretic mass ([M+3H]3+=947.1 Da) of the hinge-stabilized IgG4 peptide 219-YGPPCPPCPAPEFLGGPSVFLFPPKPK-248 (SEQ ID NO:2). The sequences of both peptides were confirmed by MS/MS analysis (data not shown).

### Example 3

### Fab-Arm Exchange in Vitro

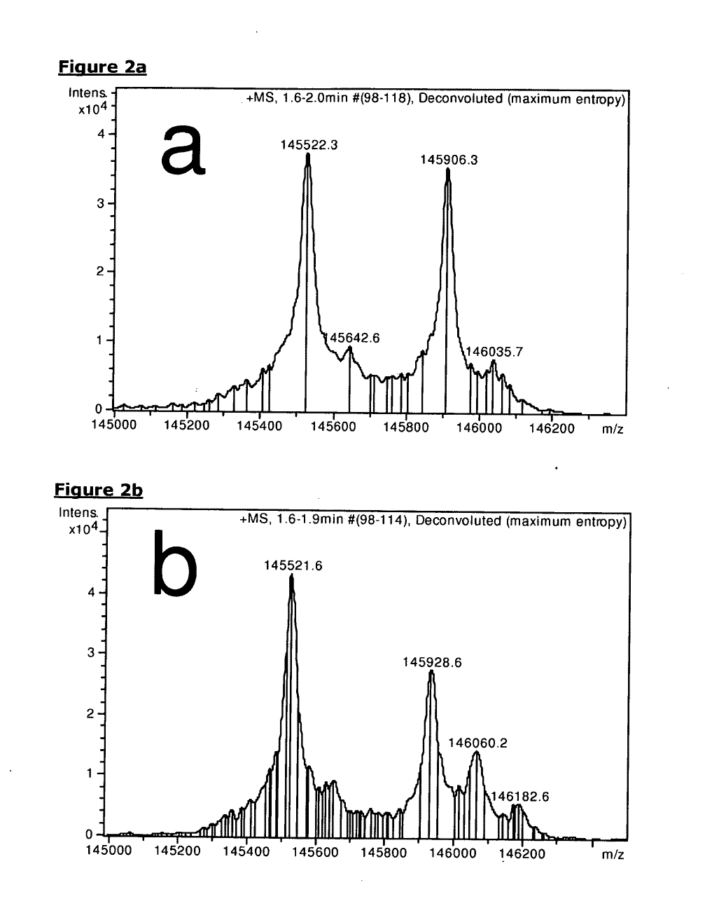

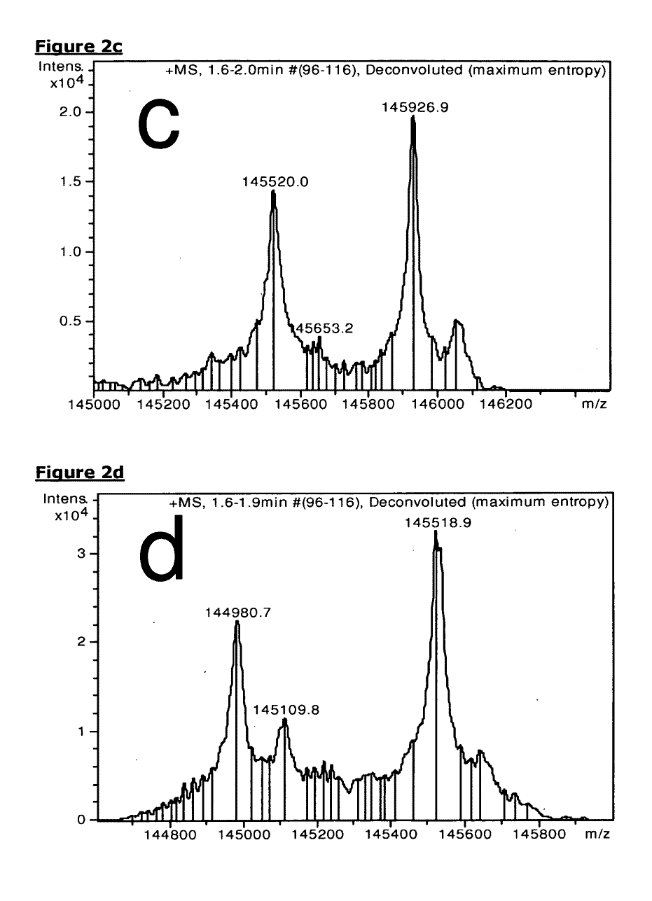

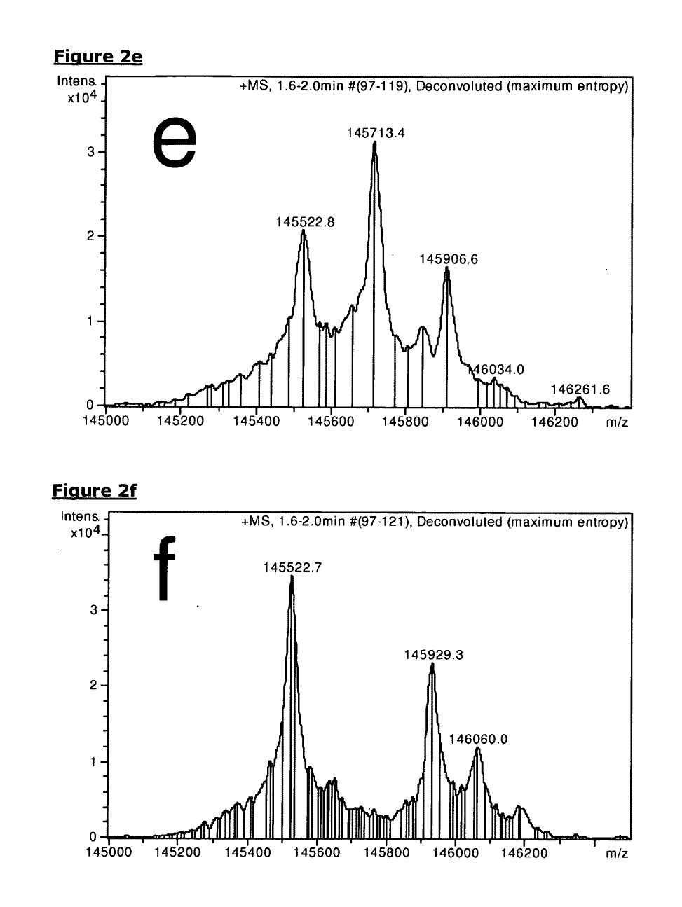

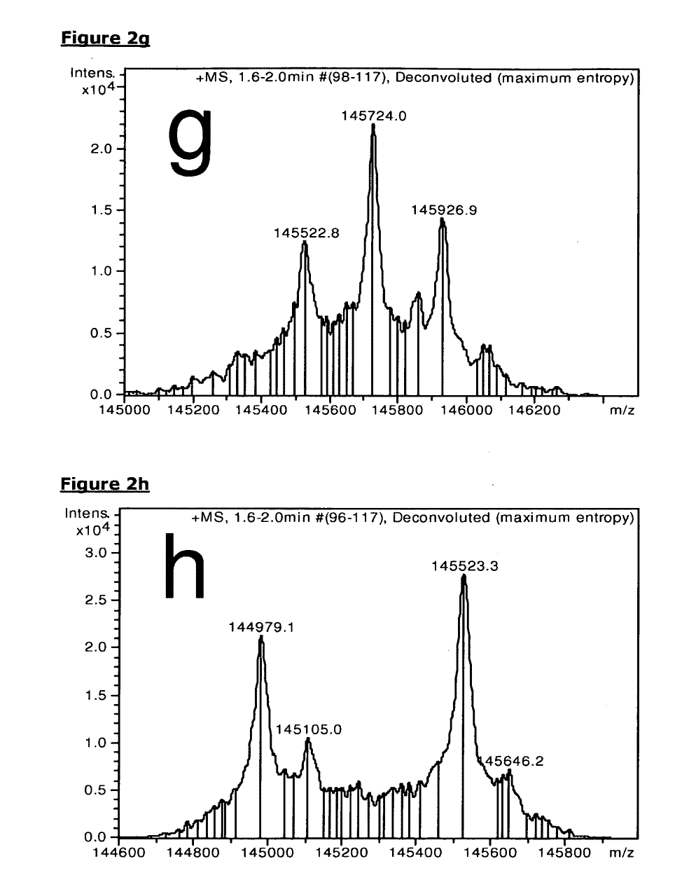

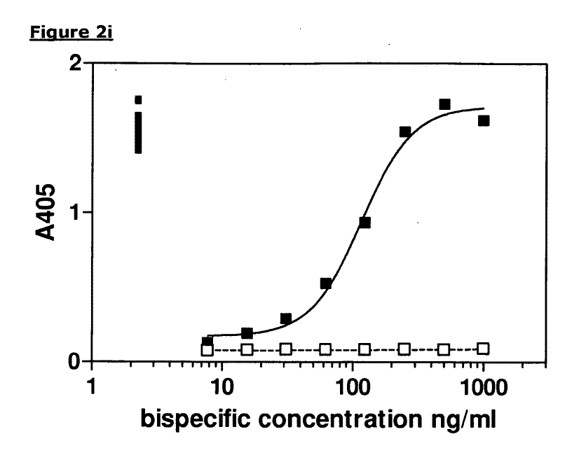

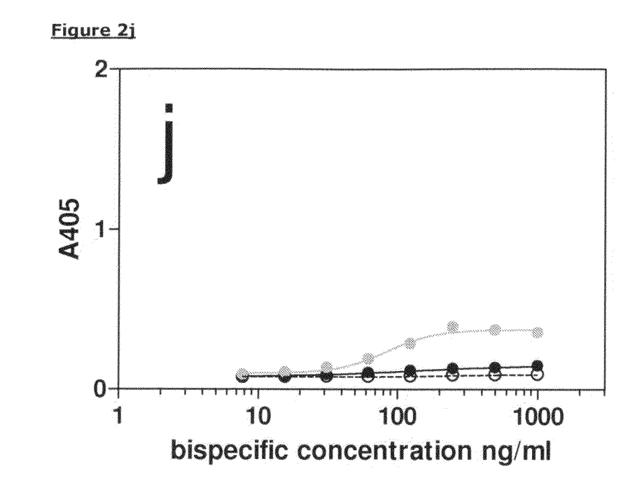

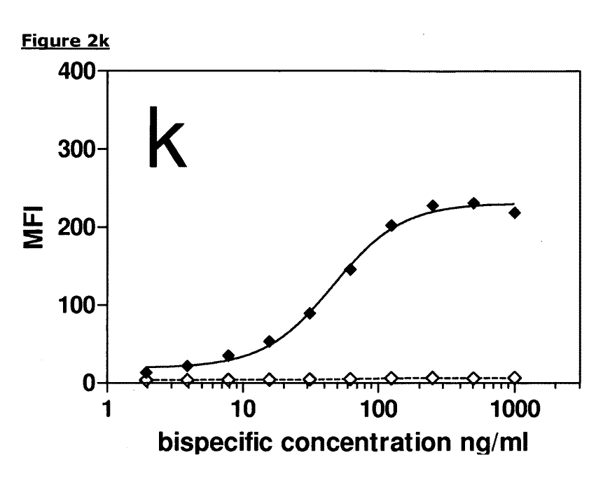

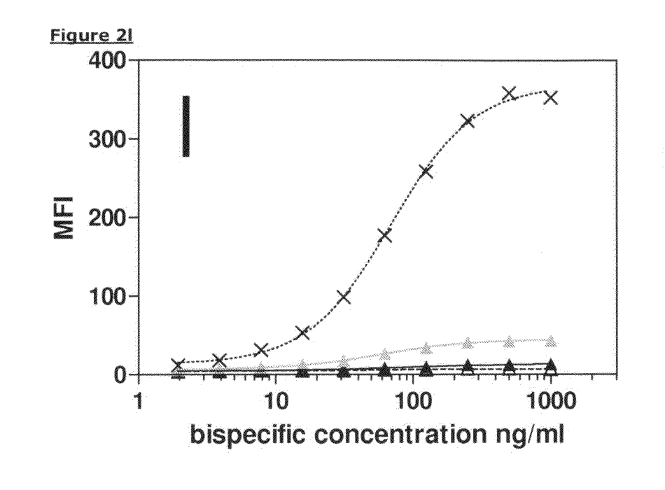

To study the effect of core-hinge stabilization alone on the exchange of Fab-arms in vitro, IgG4-EGFR, IgG4S228P-EGFR, natalizumab or gemtuzumab were mixed with IgG4-CD20 in equal amounts and incubated for 24 hrs at 37° C. in the presence or absence of 0.5 mM reduced glutathione (GSH). After deglycosylation of the mixtures, the resulting antibodies were analysed using electrospray ionization time-of-flight (ESI-TOF) mass-spectrometry. The molecular masses (of the main species without terminal lysines) of IgG4-CD20 (145.52 kDa), IgG4-EGFR (145.91 kDa), IgG4S228P-EGFR (145.93 kDa), natalizumab (145.93 kDa) and gemtuzumab (144.98 kDa) remained unchanged in the absence of GSH (FIG. 2a-d). In the presence of GSH, peaks with intermediate masses (145.71 kDa and 145.72 kDa) appeared in the mixture containing IgG4-EGFR and natalizumab, respectively, corresponding to the expected masses of CD20/EGFR and CD20/α4 integrin bispecific antibodies (FIGS. 2e and 2g). No novel peaks appeared in the presence of GSH in the mixtures containing gemtuzumab or IgG4S228P-EGFR (FIGS. 2f and 2h), suggesting that IgG4 core-hinge stabilization prevents Fab-arm exchange in vitro. Additionally, to directly demonstrate the presence of Fab-arm exchanged antibodies, bispecificity was evaluated using binding assay in which mixed bivalent antibody molecules were detected by capture on recombinant EGFR immobilized on ELISA plates, cell-surface expressed VLA-4 (α4β1 integrin) or CD33 and detection with an anti-idiotype antibody recognizing IgG4-CD20 (FIG. 2i-l). In agreement with the mass-spectrometry results, bispecific antibodies could only be detected in the wild-type IgG4 control mixture and the mixture containing natalizumab. As described previously, in vitro Fab-arm exchange required the presence of 0.5 mM GSH (4). Increasing the GSH concentration to 5 mM was able to bypass the disulfide bonds in the stabilized-hinge (FIGS. 2j and 2l), but only at the expense of antibody integrity (data not shown). The occurrence of Fab arm exchange by natalizumab is consistent with its wild-type core hinge sequence, and in addition also indicates that no other IgG4 stabilizing mutations are contained in this therapeutic antibody.

### Example 4

### Fab-Arm Exchange in Vivo

To study Fab-arm exchange in vivo, we injected equal mixtures of IgG4-CD20 with IgG1-EGFR, IgG4-EGFR, IgG4S228P-EGFR, natalizumab or gemtuzumab into immunodeficient mice. Blood samples were drawn at different time-points and bispecific antibodies were quantified using in vitro exchanged mixtures (IgG4-EGFR/IgG4-CD20 or natalizumab/IgG4-CD20) as reference standards (FIG. 3). Bispecific antibodies appeared in the blood of mice injected with mixtures containing wild-type IgG4 molecules (IgG4-EGFR and natalizumab), but not hinge-stabilized IgG4 (IgG4S228P-EGFR and gemtuzumab) or IgG1 molecules (IgG1-EGFR). Thus, core-hinge stabilization prevented IgG4 Fab-arm exchange in vivo, although we can not rule out that low-level exchange below the level of detection (<0.5% in 72 hrs) of hinge-stabilized IgG4 does occur.

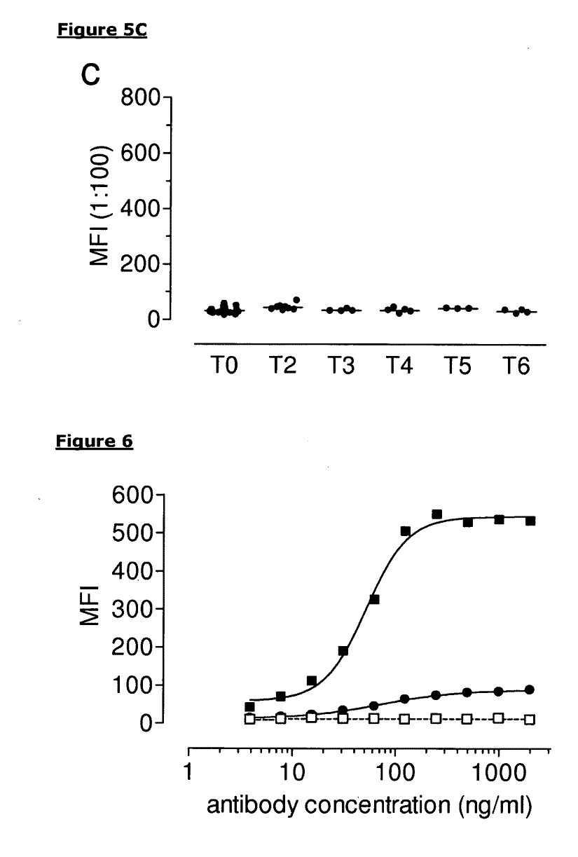

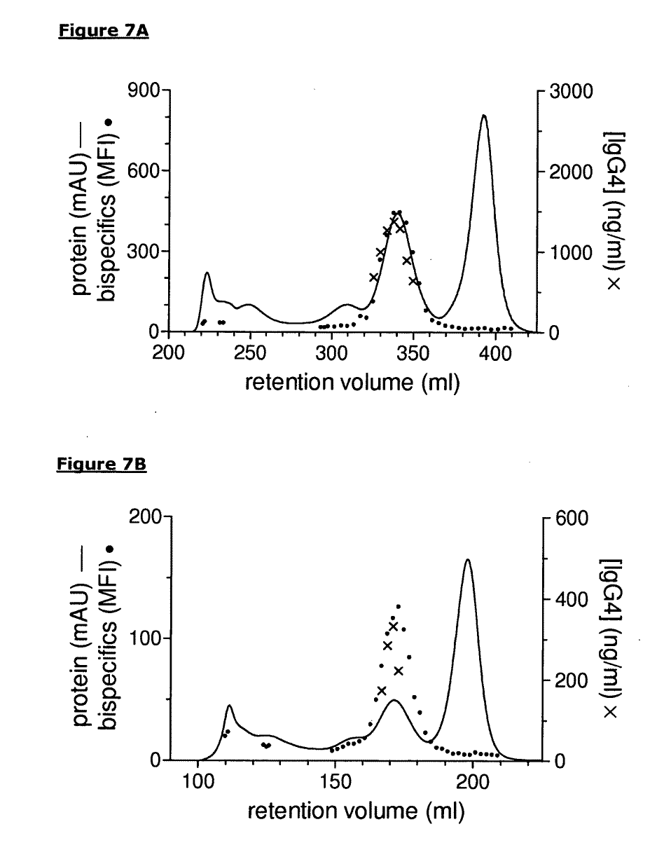

Fab-arm exchange was further studied in a therapeutic setting by investigating the dynamics of bispecific antibody formation in humans. For this, blood samples from MS patients (FIG. 4) starting natalizumab treatment (300 mg every 4 weeks) were drawn before the first infusion and at time points after subsequent infusions (FIG. 5a). To detect Fab-arm exchange of natalizumab with the heterogeneous, polyclonal IgG4 pool in these patients, we exploited the characteristic that part of human plasma IgG4 is paired with a lambda light-chain, whereas natalizumab contains a kappa light-chain. Thus using a lambda light-chain-specific detecting reagent, binding of Fab-arm exchanged natalizumab to VLA-4 on Jurkat cells was evaluated (FIG. 6). The presence of Fab-arm exchanged natalizumab could easily be demonstrated in 15 out of 16 patients, however, differences in kinetics are observed (FIG. 5b and FIG. 7). Two patients, who tested positive after 5 infusions (T5), were still negative in an earlier sample after 2 infusions (T2). For the one remaining patient, that was also negative after 2 infusions, no follow-up sample was available. The observed reactivity in all samples could be blocked by addition of an excess of exogenous natalizumab, thus confirming VLA-4 specificity (FIG. 5c). To exclude the possibility that aggregates caused the observed reactivity, two representative plasma samples were separated on size-exclusion chromatography and fractions tested for bispecific reactivity which, indeed, eluted at the expected position for monomeric IgG (FIG. 7).

1. A method of assessing the risk, for an individual, of developing an adverse event upon treatment with a therapeutic antibody which is capable of Fab-arm exchange, said method comprising the steps of:
a) providing a sample from an individual who is a candidate for treatment with said therapeutic antibody,
b) assaying said sample for the presence of circulating IgG4 antibodies that bind an antigen known or suspected to be associated with a causative agent of said adverse event, and
c) assessing, on the basis of the outcome of the assay of step b), the risk that the individual will develop said adverse event upon treatment with the therapeutic antibody, wherein the risk of development of said adverse event increases with increased level of said circulating IgG4 antibodies.

2. The method of claim 1, wherein the adverse event is an infectious disease and the antigen is an antigen of an infectious agent.

3. The method of any one of claim 1 or 2, wherein the therapeutic antibody is an IgG4 antibody.

4. The method of any one of claim 2 or 3, wherein the therapeutic antibody is an antibody that binds a molecule which is present on cells or tissues that are susceptible of being infected by said infectious agent or a molecule which is present on cells that are capable of transporting said infectious agent to a susceptible tissue.

5. The method of any one of the preceding claims, wherein the therapeutic antibody is an antibody that binds a molecule selected from the group consisting of: an integrin subunit, such as VLA-4, CD4, intercellular adhesion molecule 1 (ICAM-1), Fc gamma RI, Fc gamma RII, beta2 microglobulin, CD15, CD33 and low density lipoprotein (LDL) receptors.

6. The method of any of the preceding claims, wherein the adverse event is a viral disease and the antigen is a viral antigen.

7. The method of claim 6, wherein the adverse event is progressive multifocal leukoencephalopathy and the antigen is an antigen of the JC virus.

8. The method of any one claim 6 or 7, wherein the therapeutic antibody binds VLA4.

9. The method of claim 8, wherein the therapeutic antibody is natalizumab.

10. The method of any one of claims 7 to 9, wherein the individual is a patient suffering from multiple sclerosis or Crohn's disease.

11. The method of claim 6, wherein the adverse event is a disease caused by dengue virus, such as dengue hemorrhagic fever or dengue shock syndrome, and the antigen is an antigen of dengue virus.

12. The method of claim 11, wherein the therapeutic antibody binds Fc gamma RI, Fc gamma RII, beta2 microglobulin, CD15 or CD33.

13. The method of any one of the preceding claims, wherein the individual is an immuno-compromised individual.

14. The method of any one of the preceding claims, wherein the sample is a blood sample, such as a serum sample.

15. The method of any of the preceding claims, wherein the assay in step b) comprises a qualitative detection of the presence of said circulating IgG4 antibodies.

16. The method of any of the preceding claims, wherein the assay in step b) comprises a quantitative detection of the presence of said circulating IgG4 antibodies.

17. The method of claim 16, wherein the assessment in step c) comprises comparison of the result of the assay in step b) with a cut-off value indicative of increased risk for development of the adverse event.

18. The method of any of claims 2 to 17, further determining the presence of said infectious agent in a sample from said individual.

19. A method of assessing, for an individual who has been treated with a therapeutic antibody which is capable of Fab-arm exchange, the risk of developing an adverse event, said method comprising the steps of:
a) providing a sample from said individual,
b) assaying said sample for
1. the presence of circulating IgG4 antibodies that bind an antigen known or suspected to be associated with a causative agent of said adverse event,
or
2. the presence of bispecific antibodies having a first specificity corresponding to the specificity of the therapeutic antibody and a second specificity directed against an antigen known or suspected to be associated with a causative agent of said adverse event, and
c) assessing the risk of development an adverse event on the basis of the outcome of the assay of step b), wherein the presence of said bispecific antibodies indicates an increased risk of development of an adverse event.

20. The method of claim 19, comprising one or more of the features defined in claims 2-18.

21. A kit comprising
a) one or more of the materials required for performing the method of any one of the preceding claims, and
b) instructions describing or referring to the method of any one of the preceding claims.

22. A kit comprising:
a) an anti-human IgG4 antibody, and
b) an antigen known or suspected to be associated with a causative agent of an adverse event, preferably an antigen of an infectious agent, more preferably a virus particle or an antigen of a virus, such as a JC virus particle or an antigen of a JC virus.

23. The kit of claim 22, further comprising instructions describing or referring to the method of any one of claims 1 to 20.


SEQUENCE LISTING (2 sequences)

SEQ ID NO: 1 - protein, 27 aa, Artificial
  partial antibody sequence
YGPPCPSCPAPEFLGGPSVFLFPPKPK

SEQ ID NO: 2 - protein, 27 aa, Artificial
  partial antibody sequence
YGPPCPPCPAPEFLGGPSVFLFPPKPK